(19) United States
(12) Patent Application Publication
(10) Pub. No.: US 2019/0302127 A1
(43) Pub. Date: Oct. 3, 2019

(54) METHOD FOR DIAGNOSING TRAUMATIC BRAIN INJURY

(71) Applicant: SYSMEX CORPORATION, Kobe-shi, (JP)

(72) Inventor: Teresa LUKASZEWSKA, Kobe-shi (JP)

(73) Assignee: SYSMEX CORPORATION, Kobe-shi (JP)

(21) Appl. No.: 15/941,698

(22) Filed: Mar. 30, 2018

(51) Int. Cl. G01N 33/68; C12N 5/079

(52) U.S. Cl. CPC G01N 33/6896; C07K 16/18; G01N 33/6857; C12N 5/0622

(57) ABSTRACT

It is an object of the present invention to provide a method that can diagnose traumatic brain injury irrespective of its degree, even when it is particularly mild. Disclosed is a method for diagnosing traumatic brain injury, including the steps of: (1) bringing Glial Fibrillary Acidic Protein (GFAP) in a sample collected from a subject into contact with an anti-GFAP capture antibody to form a complex containing the GFAP and the anti-GFAP capture antibody, (2) obtaining a measured value of the GFAP by detecting the GFAP in the complex, and (3) determining whether the subject has traumatic brain injury based on the measured value, wherein the epitope of the anti-GFAP capture antibody is within the 60th to 383rd amino acid sequences in SEQ ID NO: 1.

### TECHNICAL FIELD

The present invention relates to a method and the like for diagnosing traumatic brain injury.

### BACKGROUND

Biomarkers of central nervous system (CNS) injury are not only useful in determining the severity of brain injury and cytopathology, but also can be utilized in terms of therapeutic intervention. A number of potential biochemical markers for traumatic brain injury (TBI) have been reported so far, among which GFAP (Glial fibrillary acidic protein) has attracted attention. For example, it has been reported in recent years that a significant difference was observed by measuring and comparing GFAP concentrations in specimens obtained from healthy persons and patients suspected of mild and moderate traumatic brain injury (L. Song et al., ‘Development of Digital ELISAs for the Ultrasensitive Measurement of Serum Glial Fibrillary Acid Protein and Ubiquitin C-terminal Hydrolase L1 with Clinical Utility in Human Traumatic Brain Injury’, 2017 Alzheimer's Association International Conference, poster).

### SUMMARY

The scope of the present invention is defined solely by the appended claims, and is not affected to any degree by the statements within this summary.

However, in the above report, the measurement results on patients suspected of mild traumatic brain injury and the measurement results on patients suspected of moderate traumatic brain injury are statistically analyzed in a summarized state. Therefore, whether or not healthy persons and patients suspected of mild traumatic brain injury can be stratified with high sensitivity and high specificity is unknown.

It is an object of the present invention to provide a method that can diagnose traumatic brain injury irrespective of its degree, even when it is particularly mild. Preferably, it is also an object of the present invention to provide a method that can diagnose traumatic brain injury more simply, more efficiently, and with higher sensitivity.

The present disclosure includes the following embodiments.

Item A.

A first aspect of the present invention is a method for diagnosing traumatic brain injury including the steps of:

(1) bringing GFAP in a sample collected from a subject into contact with an anti-GFAP capture antibody to form a complex containing the GFAP and the anti-GFAP capture antibody,

(2) obtaining a measured value of the GFAP by detecting the GFAP in the complex, and

(3) determining whether the subject has traumatic brain injury based on the measured value,

wherein

the epitope of the anti-GFAP capture antibody is within the 60th to 383rd amino acid sequences in SEQ ID NO: 1.

Item B.

A second aspect of the present invention is a method for diagnosing traumatic brain injury including the steps of:

(1) bringing GFAP in a sample collected from a subject into contact with an anti-GFAP capture antibody and an anti-GFAP detection antibody to form a complex containing the GFAP, the anti-GFAP capture antibody and the anti-GFAP detection antibody,

(2) obtaining a measured value of the GFAP by detecting the GFAP in the complex, and

(3) determining whether the subject has traumatic brain injury based on the measured value,

wherein

the epitope of the anti-GFAP capture antibody is within the 192nd to 201st amino acid sequences in SEQ ID NO: 1 and

the epitope of the anti-GFAP detection antibody is within the 92nd to 105th amino acid sequences in SEQ ID NO: 1.

Item C.

A third aspect of the present invention is a method for detecting traumatic brain injury including the steps of:

(1) bringing GFAP in a sample collected from a subject into contact with an anti-GFAP capture antibody to form a complex containing the GFAP and the anti-GFAP capture antibody,

(2) obtaining a measured value of the GFAP by detecting the GFAP in the complex, and

(3) determining whether the subject has traumatic brain injury based on the measured value,

wherein

the epitope of the anti-GFAP capture antibody is within the 60th to 383rd amino acid sequences in SEQ ID NO: 1.

Item D.

A test reagent for traumatic brain injury, containing

an anti-GFAP capture antibody,

wherein the epitope of the anti-GFAP capture antibody is within the 60th to 383rd amino acid sequences in SEQ ID NO: 1.

Item E.

A test reagent for traumatic brain injury, containing

an anti-GFAP detection antibody,

wherein the epitope of the anti-GFAP detection antibody is within the 60th to 383rd amino acid sequences in SEQ ID NO: 1.

Item F.

A test kit for traumatic brain injury, including

an anti-GFAP capture antibody, a solid phase, and an anti-GFAP detection antibody,

wherein the epitope of the anti-GFAP capture antibody is within the 60th to 383rd amino acid sequences in SEQ ID NO: 1, and

the epitope of the anti-GFAP detection antibody is within the 60th to 383rd amino acid sequences in SEQ ID NO: 1.

Item G.

A device for detecting traumatic brain injury, including a computer having a processor and a memory under control of the processor,

wherein a computer program for making the computer to execute steps of:

(1) obtaining a measured value of GFAP in a sample of the subject,

(2) comparing the measured value of GFAP in the sample of the subject with a predetermined standard value, and

(3) when the measured value of GFAP in the sample of the subject is lower than the predetermined standard value, determining that the subject has traumatic brain injury, is recorded in the memory.

Item H.

A computer program for making the computer including a processor and a memory under control of the processor to execute steps of:

(1) obtaining a measured value of GFAP in a sample collected from a subject,

(2) comparing the measured value of GFAP in the sample of the subject with a predetermined standard value, and

(3) when the measured value of GFAP in the sample of the subject is lower than the predetermined standard value, determining that the subject has traumatic brain injury.

### BRIEF DESCRIPTION OF DRAWINGS

FIG. 1 shows a schematic diagram of an ELISA system of Example 1;

FIG. 2 shows results of measuring the specimens with HISCL and Simoa, using combinations of antibodies of Format 1 to 3 in Example 2;

FIG. 3 is a view comparing results obtained from the same sample in the HISCL assay system using Format 2 and the Simoa assay system in Example 2;

FIG. 4 is a view comparing results obtained from the same sample in the HISCL assay system using Format 3 and the Simoa assay system in Example 2;

FIG. 5 shows results of measuring specimens (serum) obtained from patients with mild traumatic brain injury using Format 3 in Example 5;

FIG. 6 is a schematic view showing an example of a test kit;

FIG. 7 is a schematic view showing an example of a device for detecting traumatic brain injury;

FIG. 8 is a block diagram showing a hardware configuration of the device for detecting traumatic brain injury; and

FIG. 9 is a flowchart of a method for detecting traumatic brain injury using a device for detecting traumatic brain injury.

### DESCRIPTION OF EMBODIMENTS

### 1. Explanation of Terms

First, terms used in this specification, claims and abstract will be explained.

“Traumatic brain injury” is a brain injury caused by a physical impact to the head, and the degree of severity is classified from the state of consciousness at the time of injury. For example, Glasgow coma scale (GCS) of 13 to 15 points denotes mild traumatic brain injury, GCS of 9 to 12 points denotes moderate traumatic brain injury, and GCS of 8 points or less denotes severe traumatic brain injury. According to the method of the present disclosure, even mild traumatic brain injury can be diagnosed.

“Individual” is a human. The age and gender of the individual do not matter. Preferably, the individual is a living individual.

“Subject” may be an individual having some disease or an individual who does not have any disease. In addition, the subject may be an individual who received a physical impact (or possibly received a physical impact) to the head, an individual who is conscious of consciousness disorder, an individual who is determined to have consciousness disorder (or determined to possibly have consciousness disorder) in medical interview, an individual suspected of having traumatic brain injury based on the Glasgow coma scale, an individual suspected of having traumatic brain injury based on medical examination, or a person who is asymptomatic.

“Sample” includes cells, tissues, urine, blood samples and the like derived from a living body. The sample is preferably a “blood sample”.

“Blood sample” refers to blood (whole blood) collected from a subject, or serum or plasma prepared from the blood. The blood sample is more preferably serum or plasma, and further preferably serum. The type of anticoagulant used for collecting plasma is not particularly limited. The type of the blood sample of the subject used for measurement and the type of the blood sample used for determining a predetermined standard value may be the same as or different from each other, but are preferably the same as each other. When plasma is used as the blood sample, it is preferable that the plasma for determining the predetermined standard value is prepared from blood collected using the same anticoagulant as in the plasma of the subject. The blood sample may be a fresh sample or may be a preserved sample. When preserving a blood sample, it can be preserved in a room temperature environment, a refrigerated environment, or a frozen environment, but cryopreservation is preferable.

The sampling timing is preferably within 48 hours, and further preferably within 24 hours, from the time when the subject received (or the time presumed to have received) a physical impact to the head. The sample may be subjected to measurement immediately after collection or may be cryopreserved after collection and then subjected to measurement.

“Measured value of GFAP” refers to a value reflecting the amount or concentration of GFAP protein (simply referred to as “GFAP” in this specification). When the measured value is indicated by “amount”, it may be expressed on either a mole basis or a mass basis, but it is preferable to indicate the amount on a mass basis. When the value is expressed in terms of “concentration”, it may be a molar concentration or a ratio (mass/volume) of a mass per constant volume of a sample, but the value is preferably expressed in terms of a ratio of mass/volume. In addition to the above, the value reflecting the amount or the concentration may be the intensity of a signal such as fluorescence or luminescence.

“Predetermined standard value” refers to a baseline of the measured value of GFAP. The baseline can be determined based on the measured value of GFAP in a sample of an individual who does not have traumatic brain injury and/or the measured value of GFAP in a sample of an individual who has traumatic brain injury.

For example, GFAP measured values measured using samples of a plurality of individuals who have traumatic brain injury and GFAP measured values measured using samples of a plurality of individuals who do not have traumatic brain injury are obtained. Based on these multiple values, a value that can most accurately classify positive and negative can be set as a “baseline”. Here, “the value that can most accurately classify” can be appropriately set based on indices such as sensitivity, specificity, positive predictive value, negative predictive value, and the like, depending on the purpose of the examination.

For example, as one embodiment, the highest measured value among measured values of GFAP in each sample obtained from a plurality of individuals who do not have traumatic brain injury may be used as a baseline. When it is desirable to reduce the false positives as much as possible, the baseline can be suitably used.

In another embodiment, when determining the baseline based on the measured value of GFAP in the sample of an individual who has traumatic brain injury, the lowest measured value can be determined as the baseline among measured values of GFAP in samples of a plurality of individuals who have traumatic brain injury. For example, when it is desirable to reduce the false negatives, as much as possible, as in a screening test, the baseline can be suitably used.

In another embodiment, the baseline can be also set to a measured value per se of GFAP in a sample of an individual who does not have traumatic brain injury, or an average value, median value or most frequent value of a plurality of measured values of GFAP in individuals who do not have traumatic brain injury.

The baseline can also be determined based on measured values of GFAP in a plurality of samples of individuals who do not have traumatic brain injury.

In this case, [the average value of the measured values of GFAP] in the plurality of samples, preferably [a value obtained by subtracting “the value obtained by multiplying the standard deviation value of the measured values of GFAP in the plurality of samples by 1” from “the average value”], or more preferably [a value obtained by subtracting “the value obtained by multiplying the standard deviation value by 2” from “the average value”] can be used as the baseline.

Furthermore, a measured value of GFAP (one measured value may be used, or an average value, median value or most frequent value of a plurality of measured values may be used) obtained in the past before the subject which is the same subject received a physical impact to the head that caused traumatic brain injury can be used as the baseline.

When determining the baseline based on the measured value of GFAP in the sample of the individual who does not have traumatic brain injury and the measured value of GFAP in the sample of the individual who has traumatic brain injury, an average value of a measured value of GFAP in a sample of one individual who does not have traumatic brain injury and a measured value of GFAP in a sample of one individual who has traumatic brain injury can be used as the baseline. In addition, the “average value of the measured values of GFAP in the plurality of samples of individuals who do not have traumatic brain injury” and the “average value of the measured values of GFAP in the plurality of samples of individuals who have traumatic brain injury” are further averaged, and the resulting averaged value can be used as the baseline. In other embodiments, an individual who does not have traumatic brain injury and an individual who has traumatic brain injury may be grouped, and a median value of measured values of GFAP in samples of this group may be used as the baseline.

As yet other embodiments, in the method of determining the baseline, a measured value of GFAP in a sample of a healthy individual may be used instead of the measured value of GFAP in the sample of the individual who does not have traumatic brain injury.

These baselines may be determined when acquiring the measured value of GFAP in the sample of the subject, but may be determined in advance.

“A measured value of GFAP is higher than the predetermined standard value” refers to a case where the measured value of GFAP in the sample of the subject shows a value higher than the predetermined standard value. The upper limit value in this case is not particularly limited, but is preferably the highest value that can be shown in the sample of the individual.

“A measured value of GFAP is equal to or less than the predetermined standard value” means that the measured value of GFAP in the sample of the subject is equal to or lower than the predetermined standard value. The lower limit value in this case is not particularly limited, but is preferably “0”. The measured value “0” indicates that the measured value is equal to or less than the detection limit of the measurement system.

As still another embodiment, a plurality of baselines may be combined to determine whether the subject has traumatic brain injury, instead of determining whether the subject has traumatic brain injury based on the above one baseline. For example, a plurality of previously measured values of GFAP of individuals who have traumatic brain injury and individuals who do not have traumatic brain injury are divided into a plurality of numerical ranges such as “high”, “medium”, and “low”. In this case, when a measured value of GFAP in a sample of a subject is distributed in the numerical range of “high”, the individual who provided the sample can be determined to have traumatic brain injury. When a measured value of GFAP in a sample of a subject is distributed in the numerical range of “low”, the individual who provided the sample can be determined not to have traumatic brain injury. When a measured value of GFAP in a sample of a subject is distributed in the numerical range of “medium”, other examination data and medical findings may be combined to determine the presence or absence of traumatic brain injury.

“Healthy individual” is not particularly limited, but is preferably refers to an individual who does not show abnormal data in examination such as biochemical examination, blood examination, urine examination, serum examination, or physiological examination. The age and gender of the healthy individual are not particularly limited.

“A plurality of samples” is 2 or more, preferably 5 or more, and more preferably 10 or more samples. These may be samples collected from different individuals or may be a plurality of samples of the same individual collected at different times.

“A plurality of measured values” are 2 or more, preferably 5 or more, and more preferably 10 or more measured values of GFAP.

“A plurality of individuals” refers to 2 or more individuals, preferably 5 or more individuals, and more preferably 10 or more individuals.

The age, gender, etc. of a subject are not always necessarily the same as those of an individual from whom a measured value of GFAP is obtained in order to determine the baseline, but it is preferable that the individual is of the same age and/or gender as the subject.

“Anti-GFAP capture antibody” and “anti-GFAP detection antibody” (sometimes collectively referred to as “anti-GFAP antibody” in this specification) are not particularly limited as long as the antibody specifically binds to GFAP, and any of polyclonal antibodies, monoclonal antibodies, and fragments thereof (for example, Fab, F(ab)2, etc.) obtained by immunizing an animal other than a human with GFAP or a part thereof as an antigen can be used. Also, immunoglobulin classes and subclasses are not particularly limited.

Preferred examples of GFAP used as an antigen and used for preparing an anti-GFAP antibody include human GFAP having the amino acid sequence represented by SEQ ID NO: 1. The GFAP used as an antigen may be one extracted from mammalian cells by a known method or may be a recombinant protein obtained by recombinant genetic engineering technology. When a part of GFAP is used as an antigen, a fragment obtained by digesting GFAP with an enzyme or the like may be used, or a peptide having the same sequence as the amino acid sequence of a part of GFAP may be used as an antigen. The peptide can be synthesized by a known method.

“Epitope” is not particularly limited and may be a linear epitope or a conformational epitope. The number of amino acid residues constituting the epitope is not particularly limited, and is, for example, 40 or less, 35 or less, 6 to 30, 6 to 25, 8 to 20, 8 to 18, or 9 to 16.

### 2. Method for Diagnosing/Detecting Traumatic Brain Injury

In the present embodiment, a measured value of GFAP in a sample is obtained, and the measured value of GFAP is used to diagnose/detect traumatic brain injury.

(1) Step of Forming Complex

In this step, GFAP in a sample collected from a subject is brought into contact with an anti-GFAP capture antibody to form a complex containing the GFAP and the anti-GFAP capture antibody.

In this step, it is possible to use an antibody capable of specifically binding to GFAP, that is, an anti-GFAP capture antibody. “3. Test Reagent” or “4. Test Kit” as will be described later may be used.

The epitope of the anti-GFAP capture antibody is within the 60th to 383rd amino acid sequences in SEQ ID NO: 1. In one embodiment, the epitope is within preferably the 79th to 266th, more preferably the 116th to 214th, and further preferably the 192nd to 201st amino acid sequences in SEQ ID NO: 1.

The order of mixing the sample and the anti-GFAP capture antibody is not particularly limited, and these may be mixed substantially simultaneously or sequentially mixed.

It is preferred that the complex is formed at 30° C. or more. This makes it possible to detect GFAP in a shorter time. The upper limit of the temperature is not particularly limited as long as it is a temperature at which GFAP, antibody and the like are not markedly denatured, and is, for example, 50° C., and preferably 45° C.

The complex is preferably formed on a solid phase. In this case, it is possible that a complex of an anti-GFAP capture antibody and GFAP in a sample is first formed and then the complex is immobilized on a solid phase, or an anti-GFAP capture antibody is immobilized on a solid phase in advance, and the immobilized anti-GFAP capture antibody is brought into contact with GFAP in a sample to form a complex. More preferred is an embodiment in which the complex is first formed and then the complex is immobilized on a solid phase.

When a complex of an anti-GFAP capture antibody and GFAP in the sample is first formed and then the complex is immobilized on a solid phase, an anti-GFAP capture antibody modified with biotin or the like is brought into contact with GFAP in a sample to form a complex. By separately binding avidins to the solid phase in advance, the complex can be immobilized on the solid phase via binding between biotin and avidins.

When immobilizing the anti-GFAP capture antibody to the solid phase in advance, the mode of immobilization of the anti-GFAP capture antibody to the solid phase is not particularly limited. For example, the anti-GFAP capture antibody may be directly bonded to the solid phase, or the anti-GFAP capture antibody and the solid phase may be indirectly bonded with another substance interposed therebetween. Examples of the direct bond include physical adsorption and the like. Examples of the indirect bond include a bond via a combination of biotin and avidin or streptavidin (hereinafter also referred to as “avidins”). In this case, by preliminarily modifying the anti-GFAP capture antibody with biotin and previously binding avidins to the solid phase, the anti-GFAP capture antibody and the solid phase can be indirectly bonded via the bond between the biotin and the avidins. In the present embodiment, it is preferable that the bond between the anti-GFAP capture antibody and the solid phase is an indirect bond via biotin and avidins.

The material of the solid phase is not particularly limited, and it can be selected from, for example, organic polymer compounds, inorganic compounds, biopolymers, and the like. Examples of the organic polymer compound include latex, polystyrene, polypropylene, and the like. Examples of the inorganic compound include magnetic bodies (iron oxide, chromium oxide, ferrite, etc.), silica, alumina, glass, and the like. Examples of the biopolymer include insoluble agarose, insoluble dextran, gelatin, cellulose, and the like. Two or more of these may be used in combination. The shape of the solid phase is not particularly limited, and examples thereof include particles, membranes, microplates, microtubes, test tubes, and the like. Among them, particles are preferable, and magnetic particles are particularly preferable.

In this step, B/F separation for removing unreacted free components not forming a complex may be carried out after the formation of the complex, preferably after formation of the complex and before detection of a labeling substance. The unreacted free component refers to a component not constituting a complex. Examples of the unreacted free component include an anti-GFAP capture antibody not bonded to GFAP, and the like. The means of B/F separation is not particularly limited, and when the solid phase is a particle, B/F separation can be performed by recovering only the solid phase capturing the complex by centrifugation. When the solid phase is a container such as a microplate or a microtube, B/F separation can be performed by removing a liquid containing an unreacted free component. When the solid phase is a magnetic particle, B/F separation can be performed by aspirating and removing a liquid containing an unreacted free component by a nozzle while magnetically constraining the magnetic particles with a magnet. This method is preferable from the viewpoint of automation. After removing the unreacted free component, the solid phase capturing the complex may be washed with a suitable aqueous medium such as PBS.

(2) Step of Obtaining Measured Value of GFAP

In this step, a measured value of the GFAP is acquired by detecting the GFAP in the complex.

In this step, the amount or concentration of GFAP contained in the sample can be measured by detecting the complex by a method known in the art. The complex can be detected, for example, using an anti-GFAP detection antibody labeled with a labeling substance, using an unlabeled anti-GFAP detection antibody, an anti-immunoglobulin antibody labeled with a labeling substance capable of binding to the unlabeled anti-GFAP detection antibody and the like, or using an anti-immunoglobulin antibody labeled with a labeling substance capable of binding to the anti-GFAP capture antibody (unlabeled) and the like, but it is preferable to use a labeled anti-GFAP detection antibody.

When an anti-GFAP detection antibody is used, the anti-GFAP detection antibody may be used for bringing GFAP in a sample into contact with an anti-GFAP capture antibody, or it may be used after a complex containing GFAP and an anti-GFAP capture antibody is formed. In the former case, the complex formation step ((1) above) is a step of bringing GFAP into contact with an anti-GFAP capture antibody and an anti-GFAP detection antibody to form a complex containing GFAP, an anti-GFAP capture antibody and an anti-GFAP detection antibody.

The epitope of the anti-GFAP detection antibody is preferably different from the epitope of the anti-GFAP capture antibody. In one embodiment, the epitope of the anti-GFAP detection antibody is preferably within the 60th to 383rd in SEQ ID NO: 1. In a preferred embodiment, the epitope is within more preferably the 79th to 266th, and further preferably the 92nd to 105th amino acid sequences in SEQ ID NO: 1. In another preferred embodiment, the epitope is within more preferably the 257th to 377th, and further preferably the 338th to 352nd amino acid sequences in SEQ ID NO: 1.

The labeling substance used for the labeled anti-GFAP detection antibody or the labeled anti-immunoglobulin antibody is not particularly limited as long as the labeling substance generates a detectable signal. For example, it may be a substance which itself generates a signal (hereinafter also referred to as “signal generating substance”) or a substance which catalyzes the reaction of other substances to generate a signal. Examples of the signal generating substance include fluorescent substances, radioactive isotopes, and the like. Examples of the substance that catalyzes the reaction of other substances to generate a detectable signal include enzymes. Examples of the enzymes include alkaline phosphatase, peroxidase, β-galactosidase, luciferase, and the like. Examples of the fluorescent substance include fluorescent dyes such as fluorescein isothiocyanate (FITC), rhodamine and Alexa Fluor (registered trademark), fluorescent proteins such as GFP, and the like. Examples of the radioisotopes include ¹²⁵I, ¹⁴C, ³²P, and the like. Among them, an enzyme is preferable as a labeling substance, and alkaline phosphatase is particularly preferable.

The labeled anti-GFAP detection antibody is obtained by labeling an anti-GFAP detection antibody with the above labeling substance by a labeling method known in the art. Labeling may also be performed using a commercially available labeling kit or the like. As the labeled immunoglobulin antibody, the same method as the labeling of the anti-GFAP antibody may be used, or a commercially available product may be used.

In this step, by detecting a signal generated by the labeling substance of the labeled anti-GFAP antibody contained in the complex, the measured value of GFAP contained in the sample can be obtained. The phrase “detecting a signal” herein includes qualitatively detecting the presence or absence of a signal, quantifying a signal intensity, and semi-quantitatively detecting the intensity of a signal. Semi-quantitative detection means to show the intensity of the signal in stages such as “no signal generated”, “weak”, “medium”, “strong”, and the like. In this step, it is preferable to detect the intensity of a signal quantitatively or semi-quantitatively.

Methods for detecting a signal themselves are known in the art. In this step, a measurement method according to the type of signal derived from the labeling substance may be appropriately selected. For example, when the labeling substance is an enzyme, signals such as light and color generated by reacting a substrate for the enzyme can be measured by using a known apparatus such as a luminometer or a spectrophotometer.

The substrate of the enzyme can be appropriately selected from known substrates according to the type of the enzyme. For example, when alkaline phosphatase is used as the enzyme, examples of the substrate include chemiluminescent substrates such as CDP-Star (registered trademark) (disodium 4-chloro-3-(methoxyspiro[1,2-dioxetane-3,2′-(5′-chloro)tricyclo[3.3.1.13,7]decan]-4-yl)phenyl phosphate) and CSPD (registered trademark) (disodium 3-(4-methoxyspiro[1,2-dioxetane-3,2-(5′-chloro)tricyclo[3.3.1.13,7]decan]-4-yl)phenyl phosphate), and chromogenic substrates such as 5-bromo-4-chloro-3-indolyl phosphate (BCIP), disodium 5-bromo-6-chloro-indolyl phosphate, and p-nitrophenyl phosphate. Particularly preferred is CDP-Star (registered trademark). The luminescence of the substrate is preferably detected with a luminometer.

When the labeling substance is a radioactive isotope, radiation as a signal can be measured using a known apparatus such as a scintillation counter. When the labeling substance is a fluorescent substance, fluorescence as a signal can be measured using a known apparatus such as a fluorescence microplate reader. The excitation wavelength and the fluorescence wavelength can be appropriately determined according to the type of fluorescent substance used.

The detection result of the signal can be used as the measured value of GFAP. For example, when quantitatively detecting the intensity of a signal, the measured value itself of the signal intensity or the value calculated from the measured value can be used as the measured value of GFAP. Examples of the value calculated from the measured value of the signal intensity include a value obtained by subtracting the measured value of the negative control sample from the measured value, a value obtained by dividing the measured value by the measured value of the positive control sample, combinations thereof, and the like. Examples of the negative control sample include samples not containing GFAP, such as physiological saline. Examples of the positive control sample include samples containing GFAP in a predetermined amount or at a predetermined concentration.

(3) Step of Determining Traumatic Brain Injury

Next, whether a subject has traumatic brain injury is determined, based on the measured value of GFAP in the sample of the subject obtained in step (2) above. Specifically, first, the measured value of GFAP and the predetermined standard value are compared according to a known method such as a simple comparison method or a statistical test, and when the measured value of GFAP in the sample of the subject is higher than the predetermined standard value, according to the definition for determining that “a measured value of GFAP is higher than the predetermined standard value” described in the above “1. Explanation of Terms”, the subject from whom the sample is collected can be determined to have traumatic brain injury, or when the measured value of GFAP in the sample of the subject is equal to or lower than the predetermined standard value, according to the definition for determining that “a measured value of GFAP is equal to or less than the predetermined standard value” described in the above “1. Explanation of Terms”, the subject from whom the sample is collected can be determined to have traumatic brain injury. The description in the above “1. Explanation of Terms” related to “subject”, “in the sample”, “measured value of GFAP”, “predetermined standard value”, etc. can be incorporated herein.

At least one of the GFAP measured value, the comparison result between the GFAP measured value and the predetermined standard value, and the diagnosis/detection result obtained by the “Method for Diagnosing/Detecting Traumatic Brain Injury” is provided to a physician or the like to assist diagnosis of traumatic brain injury by the physician or the like. Confirmation diagnosis of traumatic brain injury can also be performed by combining other examination data and medical findings with these detection results.

### 3. Test Reagent for Traumatic Brain Injury

The present disclosure provides a test reagent for traumatic brain injury containing an anti-GFAP antibody used in the above “2. Method for Diagnosing/Detecting Traumatic Brain Injury”.

As the anti-GFAP antibody, those described in the above “1. Explanation of Terms” and “2. Method for Diagnosing/Detecting Traumatic Brain Injury” can be used.

The test reagent of this embodiment should contain at least one type of anti-GFAP antibody. In the case where the anti-GFAP antibody is a polyclonal antibody, the anti-GFAP antibody may be a polyclonal antibody obtained by immunization with one type of antigen, or may be a polyclonal antibody obtained by immunizing the same individual in parallel with two or more types of antigens. Alternatively, each polyclonal antibody obtained by inoculating two or more types of antigens into different animals respectively may be mixed. When the anti-GFAP antibody is a monoclonal antibody, the anti-GFAP may be a monoclonal antibody produced from one type of hybridoma, or may be a monoclonal antibody produced from two or more types of hybridomas, in which two or more types of a plurality of monoclonal antibodies each recognizing the same or different epitopes may be contained. Alternatively, at least one type of polyclonal antibodies and at least one type of monoclonal antibodies may be contained as a mixture.

The form of the anti-GFAP antibody contained in the test reagent is not particularly limited, and the form may be a dry state or liquid state of antiserum or ascites containing the anti-GFAP antibody. Alternatively, the form of the anti-GFAP antibody may be a dry state or aqueous solution of a purified anti-GFAP antibody, an immunoglobulin fraction containing the anti-GFAP antibody, or an IgG fraction containing the anti-GFAP antibody.

When the form of the anti-GFAP antibody is a dry state or liquid state of antiserum or ascites containing the anti-GFAP antibody, at least one of stabilizers such as β-mercaptoethanol and DTT: protective agents such as albumin; surfactants such as polyoxyethylene(20) sorbitan monolaurate and polyoxyethylene(10) octylphenyl ether; preservatives such as sodium azide; and the like may be contained. When the form of the anti-GFAP antibody is a dry state or aqueous solution of a purified anti-GFAP antibody, an immunoglobulin fraction containing the anti-GFAP antibody or an IgG fraction containing the anti-GFAP antibody, at least one of buffer components such as a phosphate buffer; stabilizers such as β-mercaptoethanol and DTT; protecting agents such as albumin; salts such as sodium chloride; surfactants such as polyoxyethylene(20) sorbitan monolaurate and polyoxyethylene(10) octylphenyl ether; and preservatives such as sodium azide may be further contained.

The anti-GFAP antibody may be provided in a state of being immobilized on a solid phase surface or the like. The solid phase and the immobilization are as exemplified in the above section of “2. Method for Diagnosing/Detecting Traumatic Brain Injury”. The solid phase is preferably magnetic beads.

### 4. Test Kit for Traumatic Brain Injury

The present disclosure provides a test kit for traumatic brain injury which is used in the above “2. Method for Diagnosing/Detecting Traumatic Brain Injury”, including the above “3. Test Reagent for Traumatic Brain Injury”.

More specifically, the test kit for traumatic brain injury includes a test reagent containing an anti-GFAP capture antibody labeled with biotin as well as a test reagent containing an anti-GFAP detection antibody labeled with a labeling substance. The labeling substance is preferably alkaline phosphatase. This embodiment may further include a solid phase, preferably a solid phase to which avidins are bonded, more preferably magnetic beads to which avidins are bonded. This embodiment may include a substrate. The substrate is preferably CDP-Star (registered trademark).

The details of the labeling substance, solid phase, and substrate are as described in “2. Method for Diagnosing/Detecting Traumatic Brain Injury”, and the description can be incorporated herein.

The test kit of the present embodiment preferably includes two types of anti-GFAP antibodies (anti-GFAP capture antibody, anti-GFAP detection antibody) which bind to different epitopes of GFAP. In this case, a complex of the anti-GFAP capture antibody, GFAP, anti-GFAP detection antibody and labeling substance is formed on the solid phase. This detection method is generally called a sandwich ELISA. In this complex, the anti-GFAP capture antibody is immobilized on the solid phase and the anti-GFAP detection antibody is directly or indirectly bonded to the labeling substance. Here, the fact that the anti-GFAP detection antibody is indirectly bonded to the labeling substance means that the labeling substance is bonded to the anti-GFAP detection antibody via an antibody or the like. Examples of the indirect bonding include a state in which a labeled antibody recognizing the anti-GFAP detection antibody is bonded to the anti-GFAP detection antibody.

In the case where the anti-GFAP capture antibody is previously bonded to the solid phase, the test kit of the present embodiment may include the solid phase on which the anti-GFAP capture antibody is immobilized, the anti-GFAP detection antibody, and the labeling substance. The anti-GFAP detection antibody and the labeling substance may be contained in separate containers or may be contained in the same container. When the labeling substance is an enzyme and the test kit further includes a substrate, it is necessary that the enzyme and the substrate be contained in separate containers. When the test kit is provided to a user, at least two types out of the solid phase, the anti-GFAP detection antibody and the labeling substance may be packed together, or they may be separately packed.

In the case where the anti-GFAP capture antibody is not previously bonded to the solid phase, the test kit of the present embodiment may include the solid phase, the anti-GFAP capture antibody, the anti-GFAP detection antibody, and the labeling substance. At least two types out of the anti-GFAP capture antibody, the anti-GFAP detection antibody and the labeling substance may be contained in the same container, or they may be contained in separate containers. When the labeling substance is an enzyme and the test kit further includes a substrate, it is necessary that the enzyme and the substrate be contained in separate containers. When the test kit is provided to a user, at least two types out of the solid phase, the anti-GFAP capture antibody, the anti-GFAP detection antibody and the labeling substance may be packed together, or they may be separately packed.

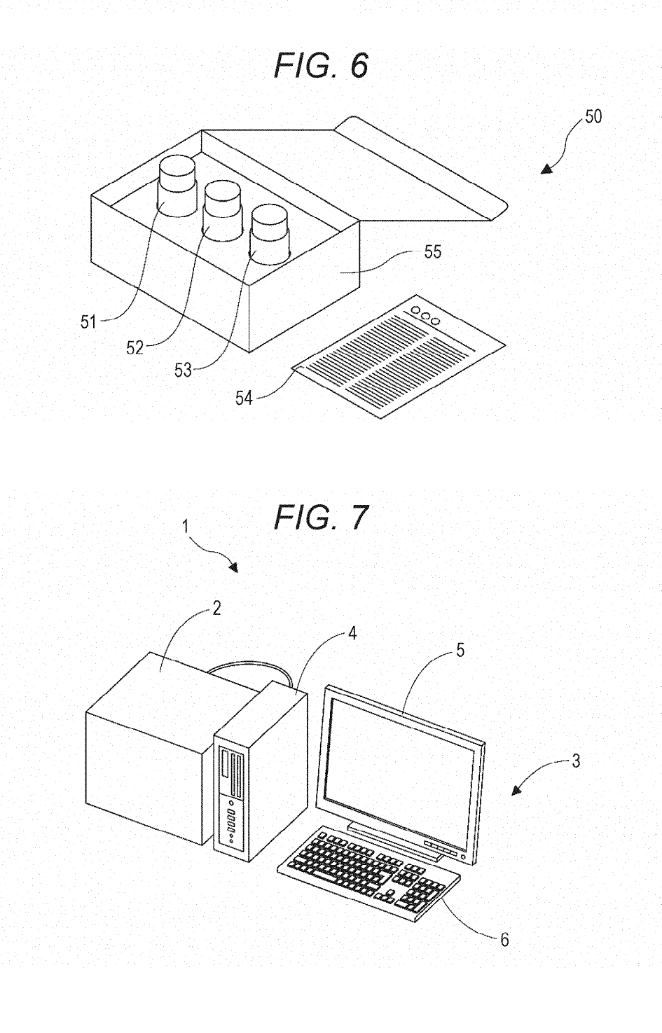

The test kit described above can be provided to a user as a kit as shown in FIG. 6. A test kit 50 for traumatic brain injury includes an exterior box 55, a first container 51 containing a plurality of magnetic particles as a solid phase, a second container 52 containing an anti-GFAP capture antibody capable of binding to the solid phase, a third container 53 containing an anti-GFAP detection antibody labeled with an enzyme, and a package insert 54 of the test kit. In the package insert 54, the handling method of the test kit, the storage conditions, the expiration date, etc. can be described. A container containing a substrate reagent, a container containing an aqueous medium for washing and the like may be packed in the exterior box 55.

The present disclosure includes a use of anti-GFAP antibody for the production of a traumatic brain injury test kit. The anti-GFAP antibody and the traumatic brain injury test kit are as described above.

### 5. Detection Device for Traumatic Brain Injury and Detection Program for Traumatic Brain Injury

Hereinafter, one embodiment of a detection device and detection program for implementing the method of the present embodiment will be described with reference to the accompanying drawings. FIG. 7 is a schematic view of a detection device 1. The detection device 1 includes a measuring device 2 and a computer system 3 connected to the measuring device 2.

The measuring device 2 measures a measured value of GFAP in a sample collected from a subject. The measuring device 2 is not particularly limited, and can be appropriately selected according to the measurement method of GFAP. The measuring device 2 of the present embodiment is a measuring device capable of detecting a signal generated by an ELISA method using, for example, a biotin-labeled anti-GFAP capture antibody, magnetic particles having avidins immobilized thereon, and an anti-GFAP detection antibody labeled with a labeling substance. This type of measuring device is not particularly limited as long as it can detect a signal based on the labeling substance used, and such a measuring device can be appropriately selected according to the type of the labeling substance.

When a biotin-labeled anti-GFAP capture antibody, a test reagent containing magnetic particles having avidins immobilized thereon, a reagent containing an anti-GFAP detection antibody labeled with a labeling substance, and a sample collected from a patient are set in the measuring device 2, the measuring device 2 executes an antigen-antibody reaction using each reagent, acquires a signal as optical information based on a labeled antibody specifically bonded to GFAP, and then transmits the obtained optical information to the computer system 3.

The computer system 3 includes a computer 4, an input unit 6 for inputting data and the like, and a display unit 5 for displaying information of a subject, detection results, and the like. Based on the optical information received from the measuring device 2, the computer system 3 obtains the measured value of GFAP in the sample collected from the subject, and detects whether or not the subject has traumatic brain injury based on the measured value of GFAP. As shown in FIG. 7, the computer system 3 may be an instrument separate from the measuring device 2, or may be incorporated in the measuring device 2.

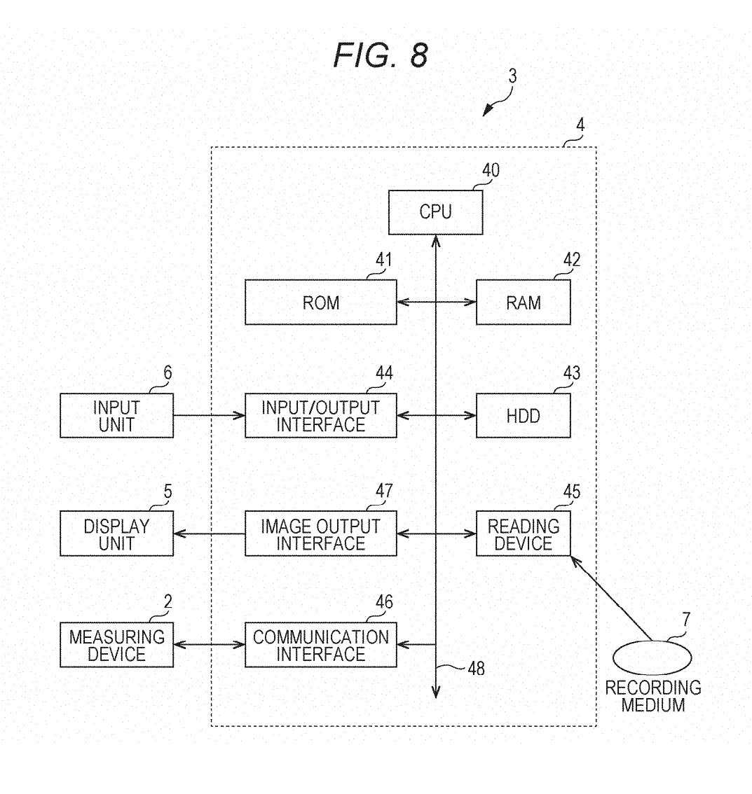

As shown in FIG. 8, the computer 4 includes a processor (CPU) 40, ROM 41, RAM 42, a hard disk (HDD) 43, an input/output interface 44, a reading device 45, a communication interface 46, and an image output interface 47, and they are connected via a bus 48 so as to enable data communication. The measuring device 2 is communicably connected to the computer 4 via a communication interface 46.

The CPU 40 controls a series of operations of each input/output part and executes a computer program stored in the ROM 41 or the hard disk 43. That is, the CPU 40 processes the optical information received from the measuring device 2 in accordance with the computer program, calculates the measured value of GFAP in the sample, and reads a predetermined standard value (cut-off value) stored in the ROM 41 or in the hard disk 43. When the measured value of GFAP is higher than the predetermined standard value, the subject from whom the sample is collected is determined to have traumatic brain injury. Then, the CPU 40 outputs the determination results and displays the results on the display unit 5.

The ROM 41 includes mask ROM, PROM, EPROM, EEPROM, and the like. As described above, a computer program (traumatic brain injury detection program) for detecting traumatic brain injury executed by the CPU 40 and data used for executing the traumatic brain injury detection program are recorded in the ROM 41. In addition to the predetermined standard value, the measured values of GFAP of the subject measured in the past and the like may be recorded in the ROM 41.

The RAM 42 includes SRAM, DRAM, and the like. The RAM 42 is used for reading the computer program recorded in the ROM 41 and the hard disk 43. The RAM 42 is also used as a work area of the CPU 40 when the CPU 40 executes these computer programs.

An operating system to be executed by the CPU 40, computer programs such as application programs (traumatic brain injury detection programs) and data used for executing the computer programs are recorded in the hard disk 43. In addition to the predetermined standard value (cut-off value), the measured values of GFAP of the subject measured in the past and the like may be recorded in the hard disk 43.

The reading device 45 includes a flexible disk drive, a CD-ROM drive, a DVD-ROM drive, and the like. The reading device 45 can read the computer program or data stored in a portable recording medium 7.

The input/output interface 44 includes, for example, a serial interface such as USB, IEEE 1394 or RS-232C, a parallel interface such as SCSI, IDE or IEEE 1284, and an analog interface including a D/A converter, an A/D converter, and the like. The input unit 6 such as a keyboard and a mouse is connected to the input/output interface 44. An operator can input various commands into the computer 4 through the input unit 6.

The communication interface 46 is, for example, an Ethernet (registered trademark) interface or the like. The computer 4 can transmit print data to a printer or the like through the communication interface 46.

The image output interface 47 is connected to the display unit 5 including an LCD, a CRT, and the like. The display unit 5 can output an image signal according to image data provided by the CPU 40. The display unit 5 displays an image according to the input image signal.

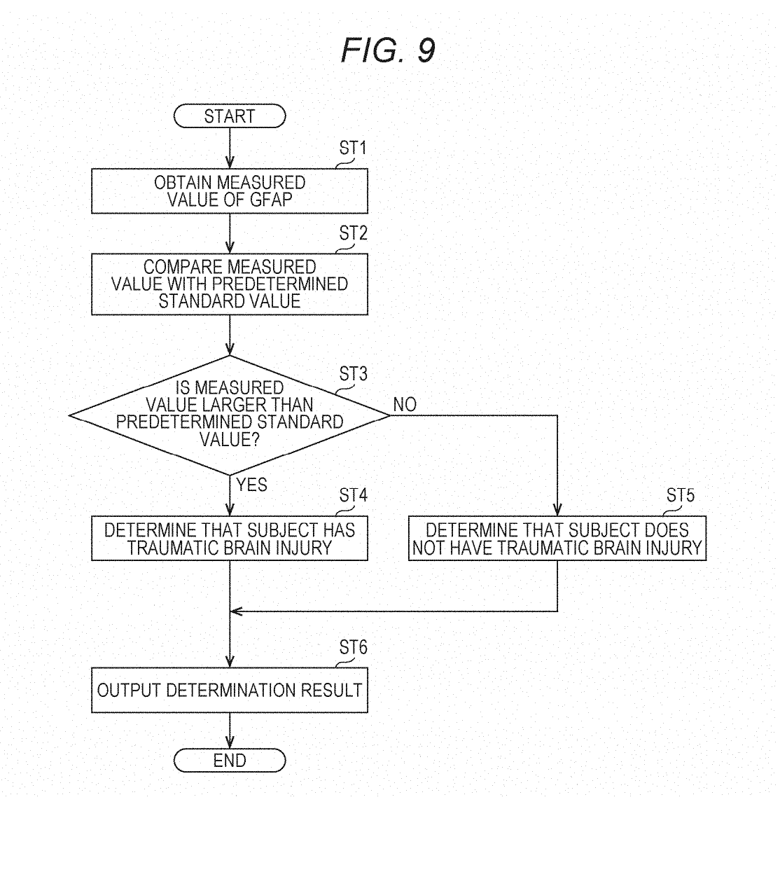

Next, with reference to FIG. 9, a detection method of traumatic brain injury executed by the detection device 1 based on the traumatic brain injury detection program will be described. First, in step ST1, upon receiving the transmission of optical information (signal) from the measuring device 2, the CPU 40 calculates the measured value of GFAP in the sample from the acquired optical information and stores the measured value in the ROM 41 or the hard disk 43. Then, in step ST2, the CPU 40 compares the obtained measured value of GFAP with the predetermined standard value (cut-off value) stored in the ROM 41 or the hard disk 43. When the measured value of GFAP is higher than the predetermined standard value (cut-off value), step ST3 proceeds to “YES”, leading to ST4 where the CPU 40 determines that the subject from whom the sample is collected has traumatic brain injury. In step ST6, the CPU 40 outputs the determination results, and displays the results on the display unit 5 or prints the results using a printer. The result that the subject from whom the sample is collected has traumatic brain injury may be stored in the ROM 41 or the hard disk 43. On the other hand, when the measured value of GFAP is the same as or lower than (equal to or less than) the predetermined standard value (cut-off value), the step ST3 proceeds to “NO”, leading to step ST5, and the CPU 40 determines that the subject from whom the sample is collected does not have traumatic brain injury. Then, in the step ST6, the CPU 40 outputs the determination results, and displays the results on the display unit 5 or prints the results using a printer. The result that the subject from whom the sample is collected does not have traumatic brain injury may be stored in the ROM 41 or in the hard disk 43.

One embodiment of the detection device for traumatic brain injury and the detection program for traumatic brain injury according to the present disclosure has been described above. However, the present disclosure is not limited to the embodiment mentioned above, and various modifications may be made without departing from the spirit of the present disclosure.

### EXPERIMENTS

Hereinafter, the present invention will be described in detail based on examples, but the present invention is not limited to these examples.

### Example 1. Antibody Screening

### Example 1-1. Primary Screening

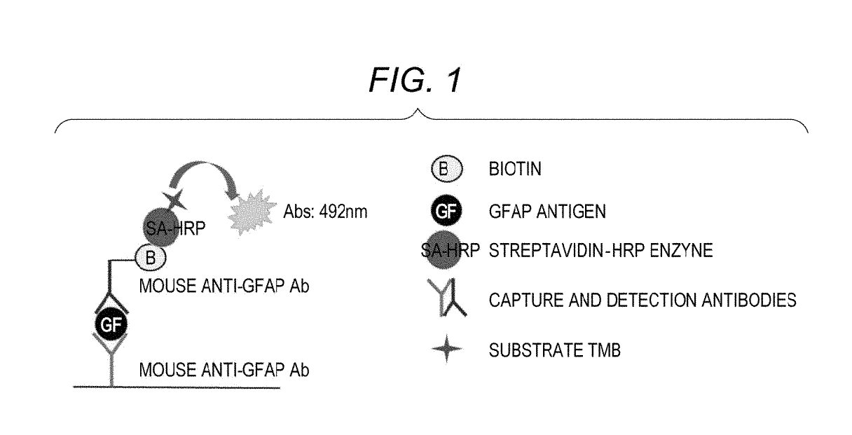

First, 17 kinds of antibodies shown in Table 1 were evaluated by ELISA system. An outline of the ELISA system is shown in FIG. 1.

TABLE 1

| | | | | |
|---|---|---|---|---|
| Ab # | Ab | ELISA | Vendor | Isotype |

1st Round Screeening (2 Antigens)

| | | | | | |
|---|---|---|---|---|---|
| 1 | mAb | KC08 | KC08 | Dx-Sys | IgG1 |
| 2 | mAb | CC10 | CC10 | Dx-Sys | IgG1 |
| 3 | mAb | CF500338 | O38 | Origene | IgG2b |
| 4 | mAb | CF500339 | O39 | Origene | IgG1 |
| 5 | mAb | CF500344 | O44 | Origene | IgG2b |
| 6 | mAb | CF500342 | O42 | Origene | IgG2b |
| 7 | mAb | CF500340 | O40 | Origene | IgG2b |
| 8 | mAb | 15C7D5D2 | BioD2 | Biolegend | IgG1 |
| 9 | Rabbit (poly) | UFP23909 | Wpol | Dr. Wang | N/A |
| 10 | mAb | UFDXM3001 | WDx | [symbol] Wang | IgG1 |
| | | | | (Dx-Sys [symbol] | |
| 11 | mAb | UFDXAB-005 | WDx | [symbol] Wang | IgG1 |
| | | | | (Dx-Sys [symbol] | |
| 12 | MCA-285 | 10152015 | En15 | EnCore | IgG1 |
| 13 | MCA-5B5 | 3182014 | En14 | EnCore | IgA |
| 14 | MCA-5C10-AP | 5313 | En13 | EnCore | IgG1 |
| 15 | Chicken (poly) | 11252014 | Enpol1 | EnCore | N/A |
| 16 | Rabbit (poly) | R15-03202014 | Enpol2 | EnCore | N/A |
| 17 | Rabbit (poly) | 178-9152015 | Enpol3 | EnCore | N/A |

[symbol]

indicates data missing or illegible when filed

Reagents used for constructing the ELISA assay system are shown below.

### Reagents/Materials:

1. Capture Ab Diluent, Carbonate-Bicarbonate Buffer pH 9.4, ThermoFisher, cat#28382

### 2. Blocking Diluent: Post Coat Buffer, Dx-Sys, Cat DXEB-003

3. Antigen Diluent: Constable-HRP—Conjugate Stabilizer—TBS, Dx-Sys, cat# DxCs-005

### 4. Detection Antibody (Biotinylated) Diluent: Biotin Antibody Dilution Buffer 1X DXEB-008

5. EZ-Link Sulfo-NHS-LC-Biotin; ThermoFlsher cat#21327
6. HRP Diluent: Constable-HRP Conjugate Stabilizwr-General—GS, cat# DXCS-002 or Constable-HRP Conjugate Stabilizer—TBS, cat# DXCS-005
7. Wash Buffer: 20×PBS Tween-20 (20×); Thermofisher, cat#28352
8. Substrate: 1-Step Ultra TMB-ELISA; THermoFisher cat#34028

### Others:

9. ELISA Plates: TheromFisher cat#434797 (from Nalge Nunc)

### 10. Desalting Column: Pierce Zeba Spin Desalting Column 7K MWCO, 2 ml

The procedure of biotinylation treatment of a labeled antibody (Detection Ab) is shown below.

1. Dialyze capture and detection Ab to PBS (Pierce Concentrator, 30K MWCO)
2. Protein concentration at A280 (NanoDrop)
3. Dilute Ab to ˜1 mg/ml with PBS
4. Calculate required volume of Biotin and Ab for 20:1 ratio

### 5. Add Biotin

### 6. Incubate 2 hrs at 4 C

7. Prepare desalting columns
7. Separate Biotin from Ab-Biotin Conjugates (Pierce Zeba Spin Desalting Column 7K MWCO, 2 ml)
8. Protein concentration at A280
9. Transfer conjugates to tubes, store in dark

Next, the measurement protocol is shown.

1. Capture Ab (2.5 ug/ml), 100 ul, over night at 4 C

### 2. Wash 4×, 300 ul

3. Block 2 hrs, RT, 250 ul

### 4. Wash 4×, 300 ul

5. GFAP Ag (5K or 10K pg/ml), 1 hr 37 C, rotation, 100 ul

### 6. Wash 4×

7. Detection Ab-Biotin (1 ug/ml), 1 hr 37 C, rotating, 100 ul

### 8. Wash 4×, 300 ul

9. SA-HRP (0.15 ug/ml), 30 min, 37 C, rotating

### 10. Wash 5×, 300 ul

11. Substrate, 100 ul, ˜20 min (in dark)
12. Stop reaction with 1 N H₂SO4, 50 ul

### 13. Read OD at 450

### Note: Use Dx-Sys ELISA Reagents

The S/N ratio values in the case of each loading the 17 kinds of antibodies on the ELISA assay system as the capture antibody (Capture Ab) and the labeled antibody (Detection Ab) and each measuring no antigen (Background), GFAP Brake Down Product (BDP) 10,000 pg/ml and GFAP Native 10,000 pg/ml are shown in Table 2.

TABLE 2

| | | S/N | S/N | Capture | Detector | |
|---|---|---|---|---|---|---|
| Capturer | Detector | GFAP-BDP | GFAP | Isotype | Isotype | |
| En14 | CC10 | 61.29 | 20.31 | IgA | IgG1 | |
| KC08 | CC10 | 54.1 | 15.71 | IgG1 | IgG1 | [symbol] |
| En14 | BioD2 | 52.14 | 13.94 | IgA | IgG1 | |
| O38 | CC10 | 51.37 | 15.37 | IgG2b | IgG1 | |
| BioD2 | CC10 | 49.96 | 14.7 | IgG1 | IgG1 | [symbol] |
| En15 | CC10 | 47.96 | 13.08 | IgG1 | IgG1 | |
| O40 | En14 | 46.63 | 13.49 | IgG2b | IgA | |
| CC10 | KC08 | 46.16 | 10.43 | IgG1 | IgG1 | [symbol] |
| O42 | En14 | 46.04 | 9.55 | IgG2b | IgA | |
| O39 | En14 | 46 | 10.33 | IgG1 | IgA | |
| O38 | En14 | 45.05 | 14.53 | IgG2b | IgA | |
| BioD2 | En14 | 44.41 | 10.94 | IgG1 | IgA | |
| En14 | O42 | 43.74 | 9.44 | IgA | IgG2b | |
| En14 | KC08 | 42.43 | 9.67 | IgA | IgG1 | |
| O39 | CC10 | 42.4 | 10.93 | IgG1 | IgG1 | |
| En15 | En14 | 42.36 | 12.34 | IgG1 | IgA | |
| CC10 | BioD2 | 41.82 | 12.62 | IgG1 | IgG1 | [symbol] |
| CC10 | O42 | 40.97 | 10.97 | IgG1 | IgG2b | |
| En14 | O38 | 40.52 | 8.09 | IgA | IgG2b | |
| CC10 | O38 | 38.21 | 8.98 | IgG1 | IgG2b | |
| KC08 | BioD2 | 38.07 | 10.65 | IgG1 | IgG1 | [symbol] |

In the above results, five combinations of capture antibody-labeled antibody were selected based on the selection criteria that the S/N ratio is high and the antibody Isotype is IgG1 (pretreatment is simple). The selected combinations are indicated by arrows in Table 2.

### Example 1-2. Secondary Screening

The secondary screening was carried out in the same manner as the primary screening, using the antibodies shown in Table 3, in addition to the antibodies (KC08, CC10, and BioD2) used in the five combinations selected in the primary screening.

TABLE 3

| | | | | |
|---|---|---|---|---|
| Ab # | Ab | ELISA | Vendor | Isotype |

2nd Round Screeening (2 Antigens)

| | | | | | |
|---|---|---|---|---|---|
| 18 | mAb | 17G12C12 | 1N | NeoClone | IgG1 |
| 19 | mAb | 10B12EB | 2N | NeoClone | IgG1 |
| 20 | mAb | 19A2D7 | 3N | NeoClone | IgG1 |
| 21 | mAb | 10D7G7 | 4N | NeoClone | IgG1 |

The results are shown in Table 4. 4N (10D7G7(H10)) showed the performance equal to or superior to the combinations of CC10/KC08 and KC08/CC10 which showed the highest performance in the primary screening.

From the above results, 10D7G7H10 (hereinafter referred to as “Ab N”) manufactured by Neoclone and CC10 (hereinafter referred to as “Ab C”) and KC08 (hereinafter referred to as “Ab K”) manufactured by Dx-Sys Inc. were selected as subjects to be evaluated in the HISCL assay system.

### Example 2. Epitope Analysis

Epitope analysis of the three antibodies (Ab N, Ab C, and Ab K) selected in the secondary screening was performed using CLIPS Precision Epitope Mapping technology from Pepscan. The results are shown in Table 5. In Table 5, the numerical value represents the amino acid number in the amino acid sequence (SEQ ID NO: 1) of human GFAP-α (Isoform 1).

TABLE 5

| Ab | Epitope | Linear/Conformational |
|---|---|---|
| Ab K | 92 QQNKALAAELNQLR 105 | Linear |
| Ab N | 192 SLEEEIRFLR 201 | Linear |
| Ab C | 338 LKDEMARHLQEYQDL 352 | Conformational |

### Example 3. Determination of Severe Traumatic Brain Injury

### 3-1. Preparation of Anti-GFAP Capture Antibody (Preparation of R1 Reagent)

Mouse monoclonal antibody IgG1 was digested with pepsin at 37° C. for 45 minutes with pepsin derived from swine gastric mucosa (manufactured by Roche) at a concentration of 2% and pH 3.5. The produced Fab′2 was purified using Superdex-200 (GE Healthcare) and HPLC system (Agilent). The purified Fab′2 was reduced at 37° C. for 1 hour with 2-Mercapto-ethyl-amine hydrochloride (Sigma) at 37° C. and pH 6.

### 3-2. Conjugate of Labeled Antibody (Preparation of R3 Reagent)

Alkaline phosphatase (ALP) was activated with N-(6-Maleimidocaproyloxy)succinimide ester (Dojindo Laboratories, Kumamoto, Japan) at 37° C. for 30 minutes. The purified product after reduction and activation was passed through a G-50 desalting column. Fab′ was added to the activated ALP at a molar ratio of 1:5 and conjugated at 2 to 8° C. for 16 to 20 hours. The conjugate was purified using 0.2 u size exclusion chromatography. The activity of ALP was measured using HISCL-800 (Sysmex).

### 3-3. Other Reagents

R2, R4 and R5 Reagents used in HISCL-800 were used.

### 3-4. Detection of GFAP Antigen Using Anti-GFAP Antibody

### 3-4-1. Measurement Protocol

GFAP in the specimen was measured with a measurement protocol of HISCL-800 manufactured by Sysmex Corporation (2-step method, specimen amount 20 ul). In this measurement protocol, the reaction between an R1 reagent containing an anti-GFAP capture antibody and a GFAP antigen is completed within 20 minutes, and preferably within 10 minutes. Similarly, the reaction between an R3 reagent containing an anti-GFAP detection antibody and a GFAP antigen is completed within 20 minutes, and preferably within 10 minutes. Any of the above-described antigen-antibody reactions is carried out in an environment at 30° C. or more. According to this measurement protocol, it completes in 17 minutes from sampling of specimen to completion of measurement.

Measurement was carried out similarly using Simoa of Quanterix, and the measurement sensitivity and reproducibility were compared.

### 3-4-2. Antibody Combination

The combinations (Format) of anti-GFAP capture antibody (Capture (R1)) and anti-GFAP detection antibody (Detection (R3)) are shown in Table 6. In Table 6, Fab′ indicates that Fab′ fragment was used as an antibody, and Whole indicates that immunoglobulin was used as an antibody.

TABLE 6

| | Format 1 | Format 2 | Format 3 |
|---|---|---|---|
| Capture(R1) | Ab K-Fab′ | Ab N-Whole | Ab N -Whole |
| | | | Ab N -Fab′ |
| Detection(R3) | Ab C-Fab′ | Ab C -Fab′ | Ab K-Fab′ |

### 3-4-3. Specimen

Specimens with severe traumatic brain injury (TBI) and healthy person specimens (Normal) were used.

### 3-5. Measurement Results

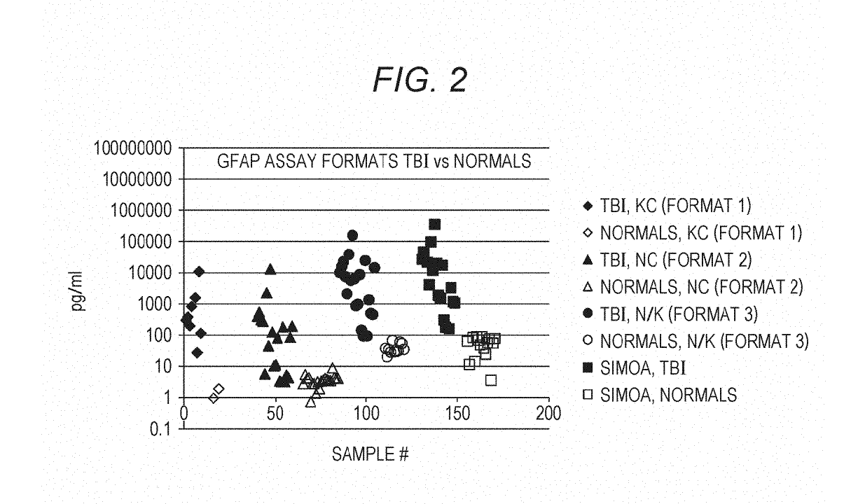

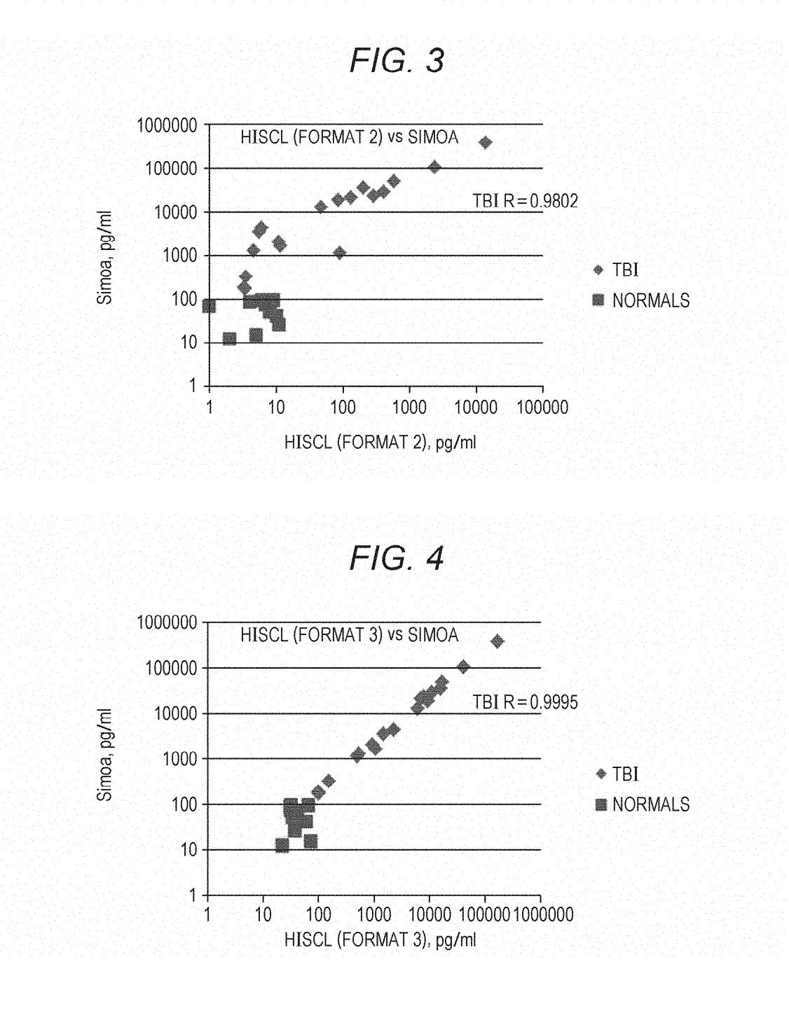

The results of measuring the specimens with HISCL and Simoa using combinations of antibodies of Format 1 to 3 are shown in FIG. 2. A view comparing results obtained from the same sample in the HISCL assay system using Format 2 and the Simoa assay system is shown in FIG. 3. A view comparing results obtained from the same sample in the HISCL assay system using Format 3 and the Simoa assay system is shown in FIG. 4. From these results, it was found that the HISCL assay system using Format 2 and Format 3 could detect GFAP with high sensitivity equal to or higher than that of Simoa.

### Example 4. Comparison of Reproducibility of HISCL Assay System and Simoa Assay System

Serum samples (=3 serum panels) containing GFAP antigens with three different concentrations were prepared. The process of repeating the measurement three times for one serum panel (that is, the sample at the same concentration) and calculating one average concentration value and one % CV value was defined as 1 run. This 1 run process was carried out 4 runs each with two HISCL-800 using the same lot on the same day. Measurement was carried out in the same manner as in Example 3, using the combination of antibodies of Format 3. The value calculated as an average value of a total of eight % CV values calculated as above was calculated as Within Run % CV, and the % CV value calculated from a total of eight average concentration values was calculated as Between Run % CV value.

The results are shown in Table 7.

TABLE 7

| | Mean | Within Run | Between Run |
|---|---|---|---|
| HISCL | pg/ml | % CV (n = 8) | % CV (n = 8) |
| Panel 5 | 22.5 | 4.00 | 6.11 |
| Panel 3 | 141.0 | 2.25 | 3.70 |
| Panel 1 | 620.6 | 1.85 | 2.91 |

The reproducibility data (each 5 runs measured with three Simoa, for 2 serum specimens, 1 plasma specimen) described in the package insert for Simoa is shown in Table 8 for comparison.

TABLE 8

| | Mean | Within Run | Between Run |
|---|---|---|---|
| Simoa | pg/ml | % CV (n = 5) | % CV (n = 5) |
| Panel 1 | 31.75 | 6.3 | 5.7 |
| Panel 2 | 317.6 | 10.9 | 13.0 |
| Panel 3 | 2.282 | 8.2 | 13.6 |

From the above results, it is understood that the HISCL assay system using the combination of antibodies of Format 3 shows high reproducibility as compared with the Simoa assay system using the combination of antibodies in Simoa.

To summarize the results of this example and Example 3, it was found that, by performing the measurement in the HISCL assay system using the combinations of Formats 1 to 3, preferably the combinations of Formats 2 to 3, and further preferably Format 3, the GFAP antigen in the specimen can be detected with high sensitivity in a time significantly shorter than the Simoa or ELISA kit. In particular, it was found that the HISCL assay system using Format 3 can detect a GFAP antigen with high reproducibility as compared with the Simoa assay system.

### Example 5. Determination of Mild Traumatic Brain Injury

### 5-1. Specimen

From patients whose value of Glasgow Coma Scale (GCS), a classification of consciousness disorder widely used worldwide, was between 13 and 15 (patients suspected of mild traumatic brain injury by a person skilled in the art), 63 specimens were collected from specimens (K2 EDTA plasma and serum) collected within 24 hours after the trauma event. When the measurement result of the CT scan was given to the specimen data, the data was also referred to. Sixty-three specimens were obtained through Trans-HitoBio, 31 specimens of which were obtained from East West Bio in Ukraine and the remaining 32 specimens were obtained from National Bioservices in Russia.

Thirty-eight specimens acquired from healthy persons were obtained from Promeddx. Of them, 20 specimens included serum and plasma specimens.

The specimens were thawed, then centrifuged at 4000 G for 7 minutes, stored at 4° C., and measured within 2 days after thawing.

### 5-2. Measurement Method

Measurement was carried out in the same manner as in Example 3, using the combination of antibodies of Format 3.

### 5-3. Measurement Results

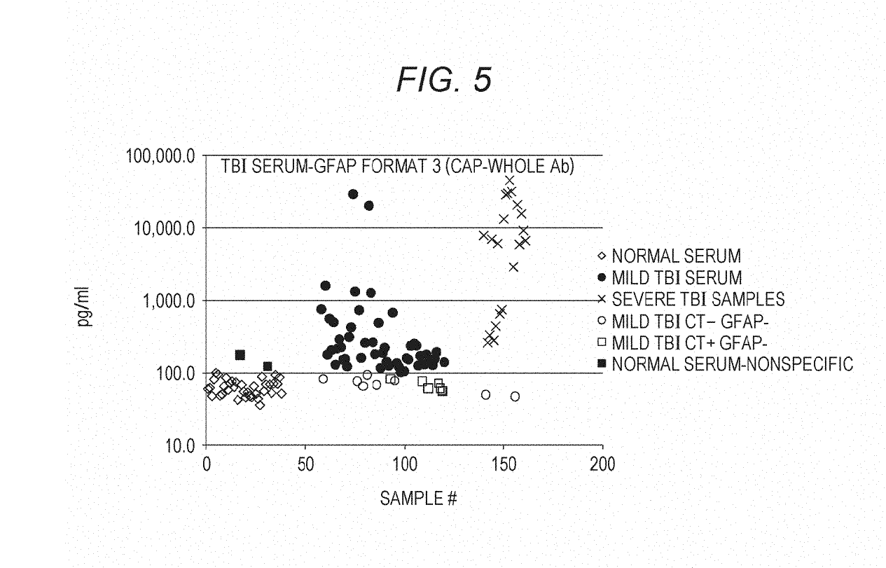

The results of measuring specimens (serum) obtained from patients with mild traumatic brain injury using Format 3 are shown in FIG. 5. A significant difference was found between healthy persons and patients with mild traumatic brain injury.

What is claimed is:

1. A method for diagnosing traumatic brain injury, comprising the steps of:
(1) bringing Glial Fibrillary Acidic Protein (GFAP) in a sample collected from a subject into contact with an anti-GFAP capture antibody to form a complex containing the GFAP and the anti-GFAP capture antibody,
(2) obtaining a measured value of the GFAP by detecting the GFAP in the complex, and
(3) determining whether the subject has traumatic brain injury based on the measured value,
wherein the epitope of the anti-GFAP capture antibody is within the 60th to 383rd amino acid sequences in SEQ ID NO: 1.

2. The method according to claim 1, wherein the traumatic brain injury is mild traumatic brain injury.

3. The method according to claim 1, wherein the epitope of the anti-GFAP capture antibody is within the 79th to 266th amino acid sequences in SEQ ID NO: 1.

4. The method according to claim 1, wherein the epitope of the anti-GFAP capture antibody is within the 116th to 214th amino acid sequences in SEQ ID NO: 1.

5. The method according to claim 1, wherein the epitope of the anti-GFAP capture antibody is within the 192nd to 201st amino acid sequences in SEQ ID NO: 1.

6. The method according to claim 1, wherein, in the forming step, the GFAP is brought into contact with the anti-GFAP capture antibody and the anti-GFAP detection antibody to form a complex containing the GFAP, the anti-GFAP capture antibody and the anti-GFAP detection antibody.

7. The method according to claim 6, wherein the epitope of the anti-GFAP detection antibody is within the 79th to 266th amino acid sequences in SEQ ID NO: 1.

8. The method according to claim 6, wherein the epitope of the anti-GFAP detection antibody is within the 92nd to 105th amino acid sequences in SEQ ID NO: 1.

9. The method according to claim 6, wherein the epitope of the anti-GFAP detection antibody is within the 257th to 377th amino acid sequences in SEQ ID NO: 1.

10. The method according to claim 6, wherein the epitope of the anti-GFAP detection antibody is within the 338th to 352nd amino acid sequences in SEQ ID NO: 1.

11. A method for diagnosing traumatic brain injury comprising the steps of:
(1) bringing Glial Fibrillary Acidic Protein (GFAP) in a sample collected from a subject into contact with an anti-GFAP capture antibody and an anti-GFAP detection antibody to form a complex containing the GFAP, the anti-GFAP capture antibody and the anti-GFAP detection antibody,
(2) obtaining a measured value of the GFAP by detecting the GFAP in the complex, and
(3) determining whether the subject has traumatic brain injury based on the measured value,
wherein
the epitope of the anti-GFAP capture antibody is within the 192nd to 201st amino acid sequences in SEQ ID NO: 1 and
the epitope of the anti-GFAP detection antibody is within the 92nd to 105th amino acid sequences in SEQ ID NO: 1.

12. The method according to claim 11, wherein the traumatic brain injury is mild traumatic brain injury.

13. The method according to claim 1, wherein, when the measured value is higher than a predetermined standard value, the subject is determined to have traumatic brain injury.

14. The method according to claim 1, wherein the complex is formed on a solid phase.

15. The method according to claim 14, wherein the B/F separation is performed after formation of the complex.

16. The method according to claim 14, wherein the solid phase is a magnetic particle.

17. The method according to claim 1, wherein the antibody includes an antibody fragment.

18. The method according to claim 1, wherein the sample is a blood sample.

19. The method according to claim 1, wherein the complex is formed at 30° C. or more.

20. A method for detecting traumatic brain injury comprising the steps of:
(1) bringing Glial Fibrillary Acidic Protein (GFAP) in a sample collected from a subject into contact with an anti-GFAP capture antibody to form a complex containing the GFAP and the anti-GFAP capture antibody,
(2) obtaining a measured value of the GFAP by detecting the GFAP in the complex, and
(3) determining whether the subject has traumatic brain injury based on the measured value,
wherein the epitope of the anti-GFAP capture antibody is within the 60th to 383rd amino acid sequences in SEQ ID NO: 1.


SEQUENCE LISTING (1 sequence)

SEQ ID NO: 1 - protein, 432 aa, Homo sapiens
MERRRITSAARRSYVSSGEMMVGGLAPGRRLGPGTRLSLARMPPPLPTRVDFSLAGALNAGFKETRASERAEMMELNDRFASYIEKVRFLEQQNKALAAELNQLRAKEPTKLADVYQAELRELRLRLDQLTANSARLEVERDNLAQDLATVRQKLQDETNLRLEAENNLAAYRQEADEATLARLDLERKIESLEEEIRFLRKIHEEEVRELQEQLARQQVHVELDVAKPDLTAALKEIRTQYEAMASSNMHEAEEWYRSKFADLTDAAARNAELLRQAKHEANDYRRQLQSLTCDLESLRGTNESLERQMREQEERHVREAASYQEALARLEEEGQSLKDEMARHLQEYQDLLNVKLALDIEIATYRKLLEGEENRITIPVQTFSNLQIRETSLDTKSVSEGHLKRNIVVKTVEMRDGEVIKESKQEHKDVM